(19) United States
(12) Patent Application Publication
(10) Pub. No.: US 2026/0228034 A1
(43) Pub. Date: Aug. 6, 2026

(54) TASK MANAGEMENT DEVICE, TASK MANAGEMENT METHOD, AND STORAGE MEDIUM

(71) Applicant: NEC CORPORATION, Tokyo (JP)

(72) Inventors: Kei SHIBUYA, Tokyo (JP); Masanori TSUJIKAWA, Tokyo (JP); Tasuku KITADE, Tokyo (JP); Yutaka UNO, Tokyo (JP); Kenji ARAKI, Tokyo (JP)

(73) Assignee: NEC CORPORATION, Tokyo (JP)

(21) Appl. No.: 19/153,025

(22) PCT Filed: Feb. 13, 2023
(86) PCT No.: PCT/JP2023/004754

§ 371 Date: Aug. 1, 2025

(51) Int. Cl. G06F 9/48

(52) U.S. Cl. CPC G06F 9/4843

(57) ABSTRACT

A task management device 1X mainly includes a task type acquisition means 15X, an identical task determination means 16X, and an output means 17X. The task type acquisition means is configured to obtain task type information, which is information indicating a task type related to each task executed in time series by a task entity that deals with a subject as a target of the task. The identical task determination means 16X is configured to determine an identical task shared by a first task entity and a second task entity based on the task type information and attribute information of the subject. The output means 17X is configured to output information related to the identical task.

### TECHNICAL FIELD

The present disclosure relates to a technical field of a task management device, a task management method, and a storage medium related to management of tasks.

### BACKGROUND

There has been known a system that manages tasks executed by an operator who performs the tasks with a person as a target. For example, Patent Literature 1 discloses a system that creates a medical act time data file for each medical practitioner from start and end times of a medical act read from a card reader. Patent Literature 2 discloses a management system that specifies and graphically displays a required time of each process of inspection-related work from the time of execution of each process.

### Citation List

### Patent Literature

Patent Literature 1: JP 2004-318751 A
Patent Literature 2: JP 2012-052850 A

### SUMMARY

### Problem to be Solved

In order to appropriately manage tasks executed by each task entity, it may be required to determine whether the executed task has been efficiently executed in an appropriate required time, specify the task to be shortened, and set a target of the required time of the task. In that case, the identical task executed by a plurality of task entities needs to be accurately determined.

In view of the problems described above, an object of the present disclosure is to provide a task management device, a task management method, and a storage medium capable of accurately determining the identical task executed by a plurality of task entities.

### Means for Solving the Problem

In one mode of the task management device, there is provided a task management device including:
a task type acquisition means for obtaining task type information, which is information indicating a task type related to each task executed in time series by a task entity that deals with a subject as a target of the task;
an identical task determination means for determining an identical task shared by a first task entity and a second task entity, based on the task type information and attribute information of the subject; and
an output means for outputting information related to the identical task.

In one mode of the task management method by a computer, the task management method includes:
obtaining task type information, which is information indicating a task type related to each task executed in time series by a task entity that deals with a subject as a target of the task;
determining an identical task shared by a first task entity and a second task entity based on the task type information and attribute information of the subject; and
outputting information related to the identical task.

It is noted that the “computer” includes any electronic device (may be a processor included in the electronic device) and may be configured by a plurality of electronic devices.

In one mode of the storage medium, there is provided a storage medium storing a program for causing a computer to execute processing including:
obtaining task type information, which is information indicating a task type related to each task executed in time series by a task entity that deals with a subject as a target of the task;
determining an identical task shared by a first task entity and a second task entity based on the task type information and attribute information of the subject; and
outputting information related to the identical task.

### Effect

An example advantage according to the present invention is to accurately determine the identical task executed by a plurality of task entities to output the determination result.

### BRIEF DESCRIPTION OF THE DRAWINGS

FIG. 1 illustrates a schematic configuration of a task management system according to a first example embodiment.

FIG. 2 illustrates an exemplary hardware configuration of a task management device.

FIG. 3 is exemplary functional blocks of the task management device according to the first example embodiment.

FIG. 4 illustrates time-series information related to tasks performed by a certain doctor from 7:00 a.m. to 10:30 a.m. on a certain day.

FIG. 5 is a correspondence table of task details represented by two types of granularity.

FIG. 6 illustrates exemplary display of an operating efficiency comparison screen image.

FIG. 7 is an exemplary flowchart to be executed by the task management device according to the first example embodiment.

FIG. 8 is a block diagram of a task management device according to a second example embodiment.

FIG. 9 is an exemplary flowchart to be executed by the task management device according to the second example embodiment.

### EXAMPLE EMBODIMENTS

Hereinafter, example embodiments of a task management device, a task management method, and a storage medium will be described with reference to the drawings.

### First Example Embodiment

### (1) System Configuration

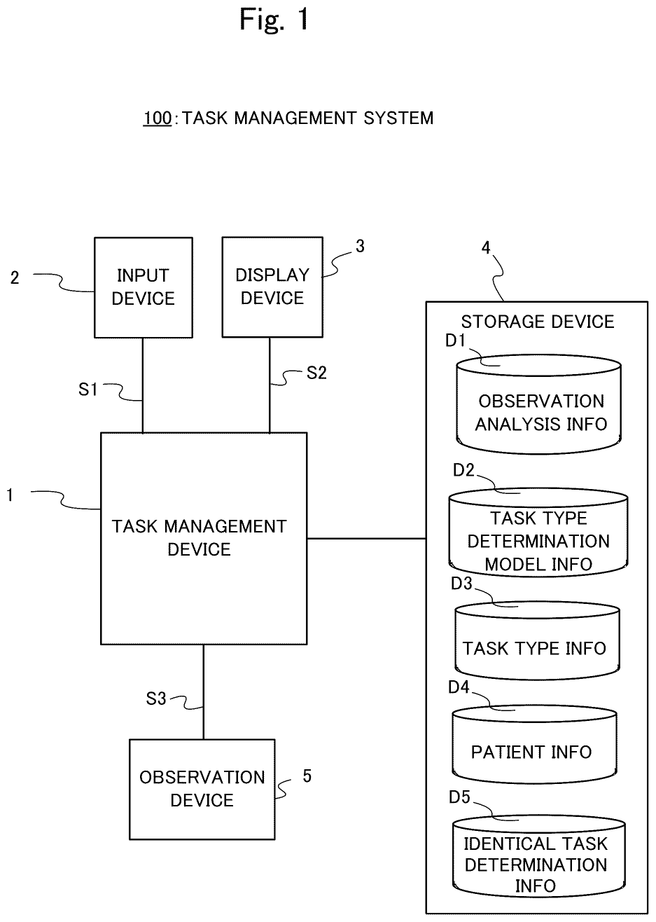

FIG. 1 illustrates a schematic configuration of a task management system 100 according to a first example embodiment. The task management system 100 collects information regarding a task executed in time series by a task entity who executes the task that deals with a subject as a target of the task, and performs display based on the collected information.

In the present example embodiment, as a representative example, a case will be described in which the “task” is a medical practice, the “task entity” who executes the task is a doctor, and the “subject” as a target of the task is a patient. The doctor as the “task entity” described above is a doctor who is a candidate for comparison regarding a required time for the executed task. The candidate mentioned above may be a doctor present in a particular hospital, or may be a doctor belonging to a particular organization (team, clinical department, etc.).

The task management system 100 mainly includes a task management device 1, an input device 2, a display device 3, a storage device 4, and an observation device 5.

The task management device 1 collects information regarding a task executed in time series by a doctor who treats a patient as a target of the tasks, determines identify of the task among a plurality of doctors, and performs display based on a result of the determination. In the display based on the result of the determination regarding the identity of the task, the task management device 1 performs display regarding comparison of the required time for the task executed by the task entity.

The task management device 1 performs data communication with the input device 2, the display device 3, and the observation device 5 via a communication network or by wireless or wired direct communication. For example, the task management device 1 receives an input signal “S1” supplied from the input device 2, an observation signal “S3” supplied from the observation device 5, and various types of information stored in the storage device 4. The task management device 1 generates a display control signal “S2” for displaying a result of the determination regarding the identity of the tasks shared by designated doctors or the like, and supplies the generated display control signal S2 to the display device 3.

The input device 2 is an interface that receives an external input (manual input). A user who inputs information using the input device 2 may be the doctor as the task entity, or may be a person who manages or supervises activities of the doctor. The input device 2 may be, for example, various types of external input interfaces, such as a touch panel, a button, a keyboard, a mouse, an audio input device, or the like. The input device 2 supplies the input signal S1 generated based on the user input to the task management device 1.

The display device 3 displays predetermined information based on the display control signal S2 supplied from the task management device 1. Examples of the display device 3 include a display, a virtual (augmented) reality terminal, and a projector.

The observation device 5 generates the observation signal S3, which is a signal obtained by observing the task performed by the doctor as the task entity, and supplies the generated observation signal S3 to the task management device 1. Examples of the observation device 5 include a sensor such as a camera including a work space of the doctor in an observation range, a medical device such as an endoscope that generates a log, and a personal computer (including various applications installed in the personal computer) operated by the doctor. The observation signal S3 includes a signal output from the sensor (including an image output from the camera), information output from the personal computer, the log output from the medical device, and the like. The information output from the personal computer includes various types of information generated by various applications installed in the personal computer (including information generated based on a user input, such as electronic medical record). The observation signal S3 includes a time stamp indicating a time point at which the observation is performed.

The observation device 5 may include a wearable terminal worn by the doctor, a microphone or the like that generates audio signals of utterance of the doctor, or a terminal operated by the doctor, such as a smartphone. For example, when the observation device 5 includes a mobile terminal such as the wearable terminal or the smartphone described above, the observation device 5 outputs, as the observation signals S3, output signals of a global navigation satellite system (GNSS) receiver, an acceleration sensor, or any other sensor for detecting biological signals built in the mobile terminal.

The storage device 4 is a memory that stores various types of information required to estimate a stress state and the like. The storage device 4 may be an external storage device, such as a hard disk connected to or incorporated in the task management device 1, or may be a storage medium, such as a flash memory. The storage device 4 may be a server device that performs data communication with the task management device 1. The storage device 4 may include a plurality of devices.

The storage device 4 mainly stores observation analysis information D1, task type determination model information D2, task type information D3, patient information D4, and identical task determination information D5.

The observation analysis information D1 is information indicating an analysis result of the observation signal S3, and is information generated by the task management device 1 based the observation signal S3. The observation analysis information D1 includes, for example, peripheral device data related to a peripheral device (e.g., medical device such as an endoscope) operated by the doctor as the task entity, and action data indicating an action of the doctor as the task entity. For example, the peripheral device data is information in which identification information indicating the medical device and operation details thereof recognized through the analysis of the observation signal S3, identification information indicating the operator (doctor) who has operated, and a time stamp are associated with each other. The action data is, for example, information in which identification information indicating an action recognized through the analysis of the observation signal S3, identification indicating the principal person (doctor) of the action, and a time stamp are associated with each other. The storage device 4 may accumulate and store the observation signal S3 before being analyzed in addition to the observation analysis information D1.

The task type determination model information D2 is information related to a task type determination model, which is a model for determining a task type of the doctor as the task entity, and is stored in the storage device 4 in advance. In the present example embodiment, as an example, the task type is assumed to be specified by a combination of a type of a task situation indicating a scene (situation) in which the task is performed and a type of task details indicating details of the task. Examples of the type of the task situation include inpatient care, outpatient care, surgery, and a medical office, and examples of the type of the task details include a medical examination, electronic medical record entry, patient explanation, and examination reservation.

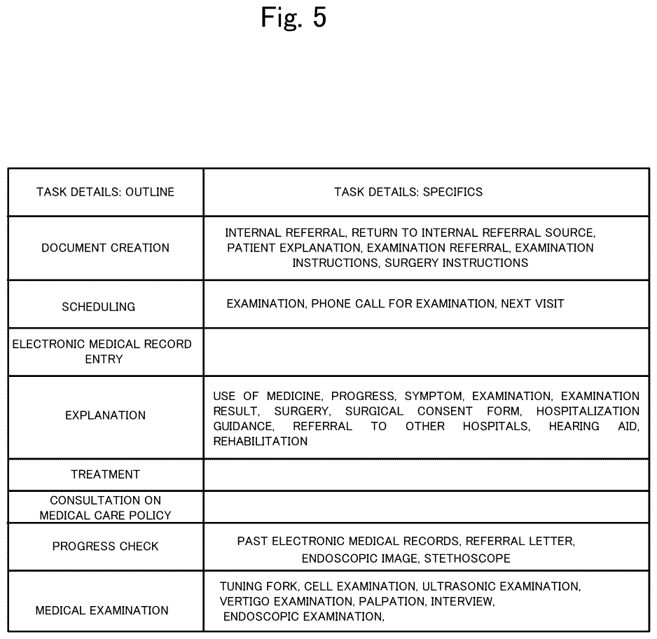

As will be described later, the task details are classified based on a plurality of levels of granularity (i.e., hierarchical categories). In this case, for example, a correspondence table indicating a correspondence relationship of types of the task details between different levels of granularity is stored in the storage device 4 in advance, and the task type determination model determines a type of the task details at the finest granularity. As a result, the task management device 1 is enabled to determine the type of the task details at each granularity based on the output of the task type determination model. The correspondence table described above will be described later with reference to FIG. 5. The task situation may also be classified based on a plurality of levels of granularity (i.e., hierarchical categories).

Here, the task type determination model will be described in a supplemental manner. In a first example, the task type determination model is one model that determines both a type of the task situation and a type of the task details. In this case, the task type determination model is a model that outputs a type of the task situation and a type of the task details when a specific type of data (e.g., identification information indicating a medical device and operation details thereof and/or identification information indicating an action), which is at least a part of the observation analysis information D1, is input. In a second example, the task type determination model includes two models (which will be referred to as a “task situation determination model” and a “task details determination model”) that individually determine a type of the task situation and a type of the task details. In this case, for example, the task situation determination model is a model that outputs a type of the task situation when a specific type of data, which is at least a part of the observation analysis information D1, is input, and the task details determination model is a model that outputs a type of the task details when a specific type of data, which is at least a part of the observation analysis information D1, is input.

The task type determination model (including the task situation determination model and the task details determination model) may be, for example, a model (including a statistical model, the same applies hereinafter) having any architecture adopted in machine learning, such as a neural network, a support vector machine, or the like, and trained based on machine learning. In this case, the task type determination model information D2 includes parameters of the task situation determination model. For example, when the model described above is a model based on a neural network such as a convolutional neural network, the task type determination model information D2 stores information regarding various parameters, such as a layer structure, a neuron structure of each layer, the number of filters and a filter size in each layer, a weight of each element of each filter, and the like. As another example, the task situation determination model may be a lookup table, an equation, or the like in which a specific type of data as at least a part of the observation analysis information D1 is associated with a type of the task situation and/or a type of the task details.

The task type information D3 is information related to a task type of each task executed by each doctor as a task entity, and is generated by the task management device 1. The task type information D3 is, for example, a database having a record for each observed task, and each record includes identification information of a doctor as a task entity, identification information of a patient as a subject of the task, a task type specified by a task situation and task details, and a time stamp indicating the time at which the task is observed.

The patient information D4 is information related to a patient for whom a doctor as a task entity performs a task. The patient information D4 is a database having a record for each patient. The patient information D4 includes, for example, patient charge information indicating a correspondence relationship between a doctor and a patient treated by the doctor (i.e., schedule of patients treated by doctors) and patient attribute information indicating an attribute of the patient.

The patient charge information is, for example, information in which identification information of a patient, a doctor who performs a task (treatment) related to the patient, and a period of time (time slot) during which the task is performed are associated with each other. The patient attribute information is information indicating any attribute of the patient, and is, for example, information regarding an operative procedure, a medical condition, a visit history, other personal medical history, age, gender, race, a job type, and the like. The patient charge information may be information stored in advance in the storage device 4, or may be information generated by the task management device 1 based on the observation signal S3, the observation analysis information D1, and/or a database or the like for managing the schedule of the patient. Likewise, the patient attribute information may be information stored in advance in the storage device 4, or may be information generated by the task management device 1 based on the observation signal S3, the observation analysis information D1, and/or a database or the like for managing the attribute of the patient.

The identical task determination information D5 is information indicating a task (which will also be referred to as an “identical task”) determined to be the same for any set (pair) of doctors. The identical task determination information D5 is generated by the task management device 1, and is stored in the storage device 4. The identical task determination information D5 is, for example, a database indicating the identical task for each pair of doctors.

The configuration of the task management system 100 illustrated in FIG. 1 is an example, and various modifications may be made to the configuration. For example, the input device 2 and the display device 3 may be configured integrally. In that case, the input device 2 and the display device 3 may be configured as a tablet terminal integrated with or separated from the task management device 1. In that case, the task management device 1, the input device 2, the display device 3, and the observation device 5 (and the storage device 4 may be included) may be configured as one smartphone to be used by the subject. The task management device 1 may include a plurality of devices. In that case, the plurality of devices included in the task management device 1 exchanges information required to execute processing allocated in advance between the plurality of devices. The task management device 1 may be connected to or incorporate an audio output device such as a speaker, and may output sound or the like relevant to the display content output by the display device 3 using the audio output device.

### (2) Hardware Configuration of Task Management Device

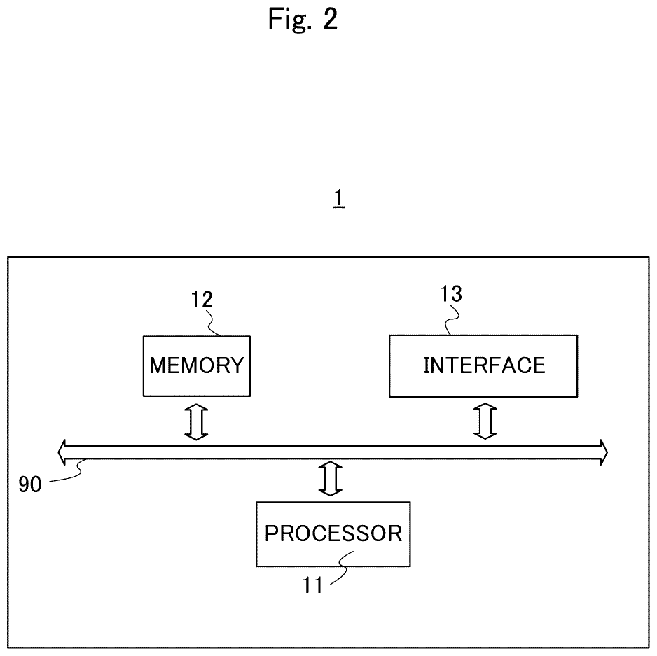

FIG. 2 illustrates a hardware configuration of the task management device 1. The task management device 1 includes, as hardware, a processor 11, a memory 12, and an interface 13. The processor 11, the memory 12, and the interface 13 are coupled to each other via a data bus 90.

The processor 11 functions as a controller (arithmetic device) that takes overall control of the task management device 1 by executing a program stored in the memory 12. Examples of the processor 11 include a central processing unit (CPU), a graphics processing unit (GPU), and a tensor processing unit (TPU). The processor 11 may include a plurality of processors. The processor 11 is an exemplary computer.

The memory 12 includes various volatile memories, such as a random access memory (RAM), a read only memory (ROM), a flash memory, and the like, and a nonvolatile memory. The memory 12 stores a program for executing processing to be performed by the task management device 1. A part of the information stored in the memory 12 may be stored in one or more external storage devices capable of communicating with the task management device 1, or may be stored in a storage medium detachable from the task management device 1. The memory 12 may function as the storage device 4. In that case, the memory 12 may store, instead of the storage device 4, at least any of the observation analysis information D1, the task type determination model information D2, the task type information D3, the patient information D4, and the identical task determination information D5.

The interface 13 is an interface for electrically connecting the task management device 1 with another device. Those interfaces may be a wireless interface such as a network adapter for wirelessly exchanging data with another device, or may be a hardware interface for connecting to the another device by a cable or the like.

The hardware configuration of the task management device 1 is not limited to the configuration illustrated in FIG. 2. For example, the task management device 1 may include at least one of the input device 2 and the display device 3. The task management device 1 may be connected to or incorporate an audio output device such as a speaker.

### (3) Identical Task Determination/Display Process

Next, processing related to determination of the identical task and display based on a result of the determination to be executed by the task management device 1 will be described. In outline, the task management device 1 determines the identical task based on the task type and the attribute of the patient, and causes the display device 3 to display the required time required for each doctor to perform the task determined as the identical task in a format that may be compared by the user. As a result, the task management device 1 allows the user to compare the required times of the identical task performed by the doctors, and suitably supports the individual doctors to specify the task to be shortened, set a target of the required time of the task, and the like.

### (3-1) Functional Blocks

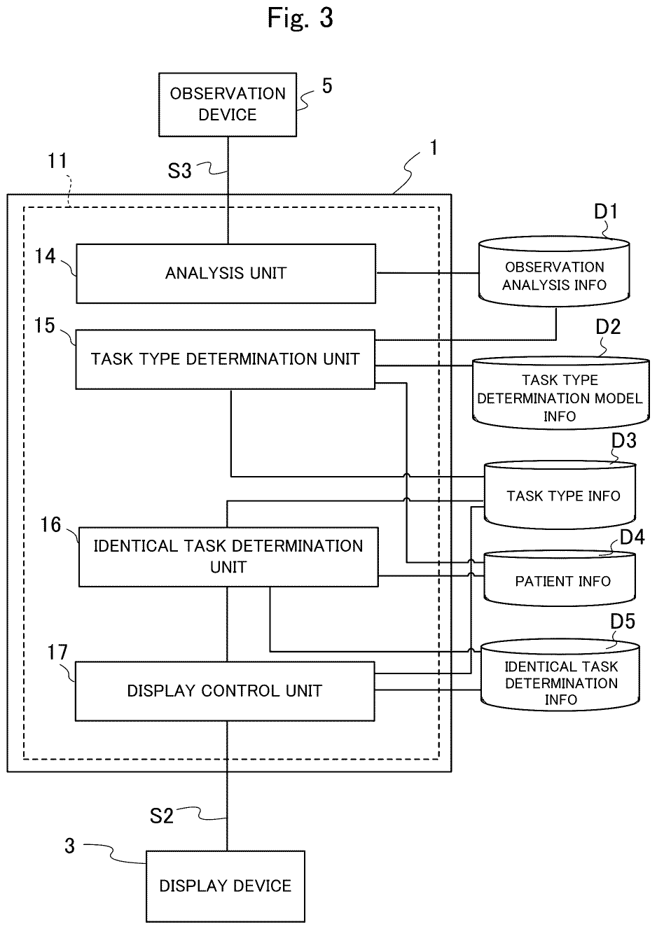

FIG. 3 is exemplary functional blocks of the task management device 1 according to the first example embodiment. The processor 11 of the task management device 1 functionally includes an analysis unit 14, a task type determination unit 15, an identical task determination unit 16, and a display control unit 17. While blocks that exchange data with each other are connected by a solid line in FIG. 3, a combination of the blocks that exchange data with each other is not limited to FIG. 3. Other functional block diagrams to be described later are in a similar manner.

The analysis unit 14 analyzes the observation signal S3 obtained in time series, and stores, in the storage device 4, the observation analysis information D1 indicating a result of the analysis of the observation signal S3 in time series. In this case, for example, the analysis unit 14 stores, in the storage device 4, the observation analysis information D1 indicating the peripheral device data related to the peripheral device operated by the doctor as the task entity and the action data indicating the action of the doctor in time series. The observation analysis information D1 includes, for example, the identification information of the doctor as the task entity and the time stamp.

The task type determination unit 15 determines, for each observed task, a doctor as a task entity of the task, a patient as a subject of the task, and a task type based on the observation analysis information D1, the task type determination model information D2, and the patient charge information in the patient information D4. In this case, the task type determination unit 15 inputs, for example, at least a part of the observation analysis information D1 to a task type determination model generated based on the task type determination model information D2, and obtains a task type output by the task type determination model. Then, the task type determination unit 15 generates the task type information D3 in which the task type output by the task type determination model, the time stamp and the identification information of the doctor identified by the observation analysis information D1, and the identification information of the patient are associated with each other. In this case, the identification information of the patient is identified based on, for example, the identification information of the doctor, the time stamp, and the patient charge information. The processing of identifying the patient as a subject of the task may be performed by the identical task determination unit 16 instead of the task type determination unit 15. In this case, the identical task determination unit 16 identifies the patient as a subject of the task based on the time stamp and the identification information of the doctor indicated by the task type information D3 and the patient charge information.

The identical task determination unit 16 determines the identical task in the pair of doctors based on the task type information D3 and the patient attribute information in the patient information D4, and stores the identical task determination information D5 indicating the task determined to be the identical task in the storage device 4.

In this case, first, the identical task determination unit 16 recognizes, based on the task type information D3, the task performed by each doctor in a predetermined target period immediately before the determination. The target period mentioned above is a period for extracting tasks to be determined whether the tasks are the identical task, and for example, it may be set to the immediately preceding work day or to the immediately preceding month for each doctor.

Next, the identical task determination unit 16 determines whether any combination of tasks (i.e., each combination corresponding to the side of the complete bipartite graph) to be compared between doctors is the identical task based on the task type and the attribute of the patient. In this case, when the number of tasks executed by the paired doctors is assumed to be “N1” and “N2” (each of N1 and N2 is an integer of 1 or more), the identical task determination unit 16 determines whether each of combinations of N1×N2 tasks indicates the identical task. When there are overlapping identical tasks in the tasks performed by the doctors, the identical task determination unit 16 may regard the tasks that are the identical task as one task, and may substantially reduce the number of tasks (i.e., N1 and N2 described above) to be compared between the doctors.

Details of the method of determining the identical task by the identical task determination unit 16 will be described later in the section of “(3-2) Identical Task Determination”.

The identical task determination unit 16 may determine the identical task for the designated pair of doctors when a request for display regarding the required time of the task is made with the designation of the pair of doctors, or may determine the identical task for any pair of doctors based on a predetermined time interval. In the former case, the identical task determination unit 16 determines the identical task for the pair of doctors designated based on the input signal S1 generated by the input device 2. In the latter case, the identical task determination unit 16 determines the identical task for all pairs selected from all the doctors whose tasks are registered in the task type information D3 (i.e., _{N}C₂ pairs assuming that the number of doctors is “N”) at the predetermined time intervals.

The identical task determination unit 16 may supply the result of the determination regarding the identical task to the display control unit 17 instead of storing it in the storage device 4 as the identical task determination information D5.

When an input of designating a pair of doctors to be subject to the comparison regarding the identical task is detected based on the input signal S1 or the like, the display control unit 17 refers to the task type information D3 and the identical task determination information D5, and generates the display control signal S2 for displaying a display screen image regarding the designated pair of doctors. Then, the display control unit 17 supplies the display control signal S2 to the display device 3 via the interface 13, thereby causing the display device 3 to display the display screen image described above. The display control unit 17 may request the identical task determination unit 16 to perform the processing of determining the identical task related to the pair of doctors designated by the user, and may generate the display control signal S2 based on a response from the identical task determination unit 16 to the request.

The components of the analysis unit 14, the task type determination unit 15, the identical task determination unit 16, and the display control unit 17 described with reference to FIG. 3 may be implemented by, for example, the processor 11 executing a program. Each component may be implemented in such a way that a required program is recorded in any nonvolatile storage medium and is installed as appropriate. At least some of those components are not limited to be implemented by software using a program, and may be implemented by, for example, a combination of any of hardware, firmware, and software. At least some of those components may be implemented using, for example, a programmable integrated circuit, such as a field-programmable gate array (FPGA), a microcontroller, or the like. In that case, a program including the components described above may be implemented using the integrated circuit. At least some of those components may include an application specific standard produce (ASSP), an application specific integrated circuit (ASIC), or a quantum processor (quantum computer control chip). As described above, the components may be implemented by various types of hardware. Other example embodiments to be described later are in a similar manner. Moreover, those components may be implemented by cooperation of multiple computers using, for example, cloud computing technology or the like.

### (3-2) Identical Task Determination

Next, a specific example of the method of determining the identical task will be described.

The identical task determination unit 16 first recognizes details of the tasks performed in time series in the target period for each doctor to be compared based on the task type information D3 and the patient charge information in the patient information D4.

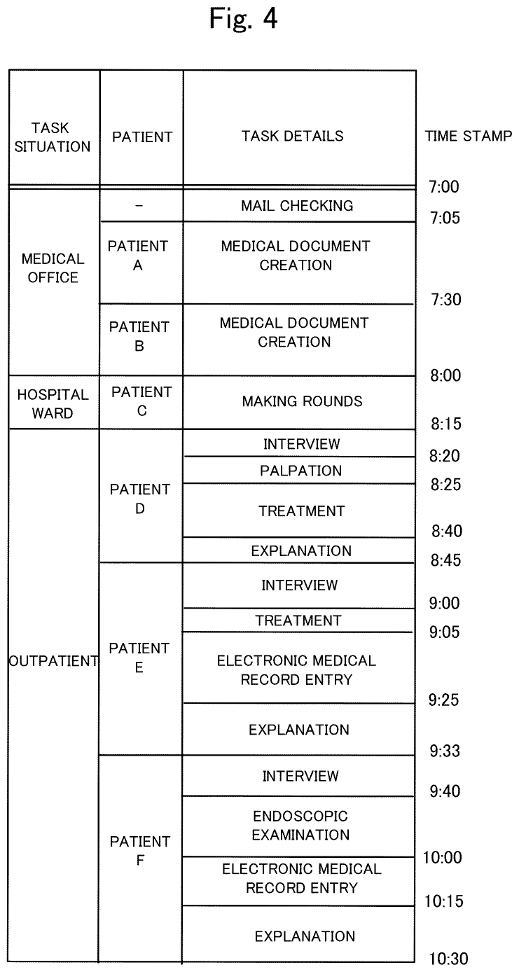

FIG. 4 illustrates time-series information recognized by the identical task determination unit 16 regarding the tasks performed by a certain doctor from 7:00 a.m. to 10:30 a.m. on a certain day. FIG. 4 illustrates a table, which is information specified by the task type information D3 and the patient information D4, in which a task type (specifically, each type of a task situation and task details) of the task, a patient as a subject of the task, and a time stamp (time) are associated with each other.

In the example of FIG. 4, the type of the task situation is any of a “medical office”, a “hospital ward” and an “outpatient”, and the patient as a subject of the task is any of a “patient A”, a “patient B”, a “patient C”, a “patient D”, a “patient E”, and a “patient F”. The type of the task details is any of “mail checking”, “medical document creation”, “making rounds”, “interview”, “palpation”, “treatment”, “explanation”, “electronic medical record entry”, and “endoscopic examination”. A period of time (i.e., start time and end time) of each task is specified by a time stamp.

In this manner, the identical task determination unit 16 executes processing of specifying the task type, the patient, and the period of time (time slot) for each task for each doctor based on the task type information D3 and the patient information D4.

Next, the identical task determination unit 16 determines, as the identical task, the task having the identical task type (i.e., type of the task details and task situation) and the identical patient attribute in the pair of doctors to be compared.

Here, a method of determining presence or absence of commonality in the type of the task details in consideration of the granularity will be described.

When the granularity of the task details to be used to determine whether the task details are common is set, the identical task determination unit 16 determines the presence or absence of the commonality in the task details by comparing the type of the task details at the set granularity. The set granularity described above is granularity selected from a plurality of levels of granularity that may be set for the type of the task details. In this case, for example, a correspondence table of the task details for each granularity is stored in advance in the storage device 4, and the type of the task details at the finest granularity is recorded as a type of the task details in the task type information D3. Then, the identical task determination unit 16 recognizes the type of the task details at the set granularity based on the correspondence table described above and the type of the task details at the finest granularity indicated by the task type information D3. The granularity of the task details to be used to determine whether the task details have commonality may be selected based on the user input or the like, or may be predetermined granularity selected in advance.

FIG. 5 is a correspondence table of the task details represented by two types of granularity. The correspondence table illustrated in FIG. 5 includes “task details: outline” and “task details: specifics”, and “task details: specifics” represent types of the task details at granularity finer than that of “task details: outline” (i.e., subdivided types). For example, “document creation” in “task details: outline” is relevant to “internal referral”, “return to internal referral source”, “patient explanation”, “examination referral”, “examination instructions”, “surgery instructions”, and the like in “task details: specifics”. “Scheduling” in “task details: outline” is relevant to “examination”, “phone call for examination”, “next visit”, and the like in “task details: specifics”. “Explanation” in “task details: outline” is relevant to “use of medicine”, “progress”, “symptoms”, “examination”, “examination result”, “surgery”, “surgical consent form”, “hospitalization guidance”, “referral to other hospitals”, “hearing aid”, “rehabilitation”, and the like in “task details: specifics”. “Progress check” in “task details: outline” is relevant to “past electronic medical records”, “referral letter”, “endoscopic image”, and the like in “task details: specifics”. “Medical examination” in “task details: outline” is relevant to “tuning fork”, “cell examination”, “ultrasonic examination”, “vertigo examination”, “palpation”, “interview”, “endoscopic examination”, “vernier caliper measurement”, and the like in “task details: specifics”.

“Electronic medical record entry”, “treatment”, and “consultation on medical care policy” in “task details: outline” are examples that may not be subdivided, and the content of “task details: specifics” is the same as that of “task details: outline”, thereby being blank here.

While the correspondence table indicates correspondence of the task details at two types of granularity in the example of FIG. 5, it may indicate correspondence of the task details at three or more types of granularity. When the task type representing the type of the task details at each granularity is recorded in the task type information D3, the correspondence table described above is not necessarily stored in the storage device 4. In that case, for example, the task type determination model is a model for determining a type of the task details at each granularity, and the task type determination unit 15 generates the task type information D3 based on an output of such a task type determination model.

Also when there is a plurality of levels of granularity in the type of the task situation and the granularity of the task situation to be used to determine commonality in the task situation is set, the identical task determination unit 16 determines the commonality in the task situation by the same method as the method of determining the commonality of the type of the task details described above.

Next, a method of determining commonality in the patient attribute will be specifically described.

A type of the patient attribute to be used to determine whether the patient attribute have commonality is one or a plurality of types of attributes, which may be predetermined or designated by the user. For example, the identical task determination unit 16 determines that patients having the same (including minor differences that may be regarded as substantially the same) age, personal medical history, number of visits, and medical condition (or treatment difficulty level) have a common attribute.

The identical task determination unit 16 may determine a type of the patient attribute to be determined whether it has commonality based on the type of the task details. As a result, the identical task determination unit 16 may more accurately determine the identical task in consideration of the patient attribute that affects the task.

For example, in a case of “document creation” as task details, the identical task determination unit 16 determines whether the patient attribute has commonality based on the number of similar cases and/or a severity level of the medical condition of the patient. As another example, in a case of “scheduling” as task details, the identical task determination unit 16 determines whether the patient attribute has commonality based on presence or absence of an occupation (regular job) and/or age of the patient. As still another example, in a case of “electronic medical record entry” as task details, the identical task determination unit 16 determines whether the patient attribute has commonality based on at least one of the number of similar cases of the medical condition of the patient, the number of medical examinations, the number of examination sites, and a progress change. As still another example, in a case of “treatment” as task details, the identical task determination unit 16 determines whether the patient attribute has commonality based on a treatment site and/or a severity level. As still another example, in a case of “consultation on medical care policy” as task details, the identical task determination unit 16 determines whether the patient attribute has commonality based on the number of similar cases of the medical condition of the patient. As still another example, in a case of “progress check” as task details, the identical task determination unit 16 determines whether the patient attribute has commonality based on at least one of the number of medical examinations, a severity level, and the number of examination sites. As still another example, in a case of “medical examination” as task details, the identical task determination unit 16 determines whether the patient attribute has commonality based on a medical examination site and/or a severity level.

In a case of comparing the types of the task details of the tasks shared by doctors in determination of the identical task, the identical task determination unit 16 may compare the types of the task details at the coarsest granularity (i.e., at the highest conception), and may exclude the tasks having different task details at the coarsest granularity from the candidates for the identical task. As a result, the tasks that are not obviously the identical task may be efficiently excluded from the candidates for the identical task to reduce the calculation amount without comparing the task details at a detailed level (i.e., at finer granularity).

### (3-3) Display Screen Image

Next, a specific example of the display screen image displayed on the display device 3 by the display control unit 17 will be described.

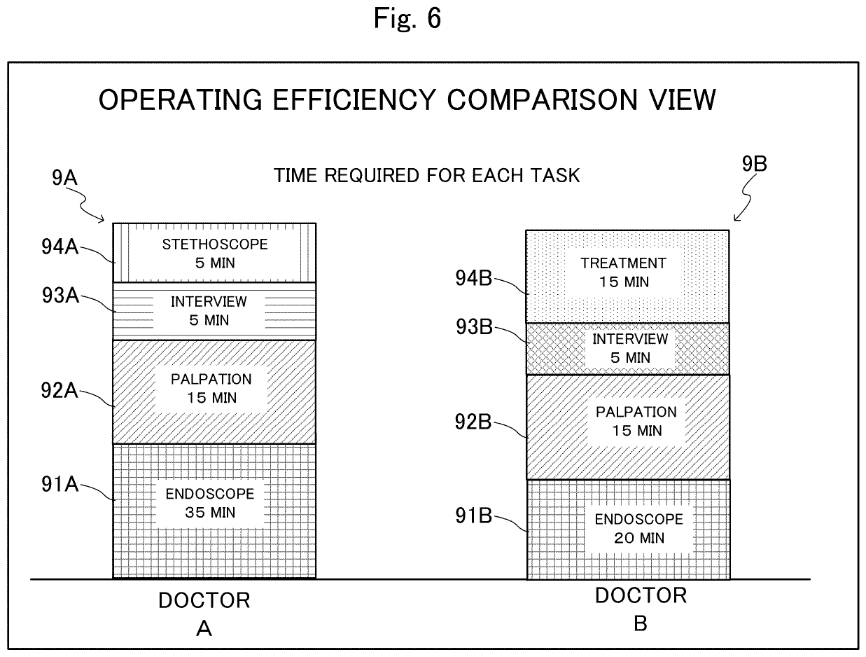

FIG. 6 is exemplary display of an operating efficiency comparison screen image, which is a display screen image for comparing the operating efficiency of any two doctors (doctor A and doctor B in this case) designated by the user. In this case, the display control unit 17 generates the display control signal S2 of the operating efficiency comparison screen image based on the identical task determination information D5 or the determination result supplied from the identical task determination unit 16, and supplies the generated display control signal S2 to the display device 3 via the interface 13, thereby causing the display device 3 to display the operating efficiency comparison screen image.

For example, the display control unit 17 displays an input screen image for selecting a doctor to be compared or the like before displaying the operating efficiency comparison screen image, and receives designation of the doctor A and the doctor B to be compared based on a user input (i.e., external input) on the input screen image using the input device 2. Through the input screen image described above, the display control unit 17 may further receive, in addition to the selection of the doctors to be compared, granularity of the task details set in the determination of the identical task, a target period for extracting the task to be determined as to whether it is the identical task, and the like.

The display control unit 17 provides, on the operating efficiency comparison screen image, a bar graph 9A representing a time required for each task by the doctor A, and a bar graph 9B representing a time required for each task by the doctor B. The bar graph 9A includes elements 91A to 94A relevant to four tasks (endoscope, palpation, interview, and stethoscope) performed by the doctor A, and the bar graph 9B includes elements 91B to 94B relevant to four tasks (endoscope, palpation, interview, and treatment) performed by the doctor B.

In this case, the display control unit 17 recognizes the tasks performed by the doctors A and B within the target period based on the task type information D3, and calculates a required time for each task. Here, when there are overlapping identical tasks performed by the same doctor in the tasks performed by the individual doctors, the display control unit 17 regards the overlapping tasks as one task. In that case, the display control unit 17 calculates an average of the required times of the overlapping tasks, and displays the overlapping tasks as a single element in the bar graph.

A method of determining the identical task by the same doctor is the same as the method of determining the identical task in the tasks shared by the doctors by the identical task determination unit 16 described above. For example, in the example of FIG. 4, the task (8:15 to 8:20) directed to the patient D, the task (8:45 to 9:00) directed to the patient E, and the task (9:33 to 9:40) directed to the patient F, which have commonality in the task type as they have the task situation of “outpatient” and the task details of “interview”, will be considered. In this example, when it is determined that the attributes (age, personal medical history, etc.) of the patient D, patient E, and patient F have commonality, the display control unit 17 determines that the three tasks described above are the identical tasks, calculates an average of the required times of those tasks, and displays one element having a length equivalent to the calculated average of the required times as a bar graph element collectively representing those tasks.

In the exemplary display of FIG. 6, the display control unit 17 recognizes, based on the identical task determination information D5, the tasks shared by the doctor A and the doctor B determined to be the identical tasks, and displays the bar graph elements relevant to the tasks determined to be the identical task in the same color. For example, since the identical task determination unit 16 determines that the tasks relevant to the element 91A and the element 91B (i.e., tasks related to the endoscope) are the identical tasks, the display control unit 17 displays the element 91A and the element 91B in the same color. Likewise, since the identical task determination unit 16 determines that the tasks relevant to the element 92A and the element 92B (i.e., tasks related to the palpation) are the identical tasks, the display control unit 17 displays the element 92A and the element 92B in the same color. As a result, the display control unit 17 may allow the user to accurately recognize the identical task shared by the doctors to be compared.

On the other hand, since the identical task determination unit 16 determines that the tasks relevant to the element 93A and the element 93B (i.e., tasks related to the interview) are not the identical tasks, the display control unit 17 displays the element 93A and the element 93B in different colors. In this case, for example, the identical task determination unit 16 determines that the tasks relevant to the element 93A and the element 93B are not the identical tasks as the task situations or the target patient attributes are different while they have commonality in the task details “interview”. As described above, the identical task determination unit 16 determines the identical task in consideration of the target patient attribute and the like in addition to the commonality in the task details, whereby only the tasks that may be compared in the review of the operating efficiency and the like may be accurately presented as the identical task. Likewise, the display control unit 17 displays the element 94A and the element 94B in colors different from those of other elements as there is no pair of the identical task.

The display control unit 17 may clearly indicate whether it is the identical task by connecting the elements of the identical task by an arrow or the like, or by indicating the elements of the identical task by a sentence, instead of clearly indicating whether it is the identical task by color coding. As described above, the display control unit 17 may indicate whether it is the identical task on the operating efficiency comparison screen image in any display form. The display control unit 17 may hide the elements of the tasks that are not the identical tasks (elements 93A, 93B, 94A, and 94B in FIG. 6) in the bar graphs 9A and 9B, and may display the bar graphs 9A and 9B including the elements of the tasks determined to be the identical tasks (elements 91A, 91B, 92A, and 92B in FIG. 6). As a result, the time required for the tasks, which are the identical tasks, may be more precisely presented to the user.

Here, an effect of displaying the determination result of the identical task shared by the doctors will be described in a supplemental manner.

For example, when the user is a doctor, it becomes possible to specify a task to be shortened and to set a target time required for the task by comparing the time required for the identical task between the user and another doctor. When the user is a person who manages or supervises activities of doctors in a team, it becomes possible to grasp strengths and weaknesses in the tasks to help each other in team medical care by comparing the time required for the identical task among the doctors in the team. A problem related to medical income and expenditure may be specified from the required task time of each doctor.

By making comparison between an experienced doctor and a young doctor, it may be utilized to set an issue to be addressed by the young doctor or the experienced doctor. For example, when the young doctor requires 30 minutes and the experienced doctor requires 15 minutes for a certain task, it is highly likely that the required time for the young doctor may be shortened by accumulating experience, and thus no issue to be addressed by the young doctor is set for that task. On the other hand, when the young doctor requires 15 minutes and the experienced doctor requires 30 minutes for a certain task, the time required for the experienced doctor is longer than that required for the young doctor with less experience, and thus the task is set as an issue to be addressed by the experienced doctor. In this case, the average required time of the young doctor regarding the task is set as a target required time of the experienced doctor regarding the task.

By making comparison among young doctors, it may be utilized to set an issue to be addressed by the young doctors. For example, when a first young doctor requires 30 minutes and a second doctor having the same years of experience as the first young doctor requires 15 minutes for a certain task, the task is set as an issue to be addressed by the first young doctor. In that case, for example, the average required time of the young doctors regarding the task is calculated, and the calculated average required time is set as a target required time of the first young doctor regarding the task.

### (3-4) Processing Flow

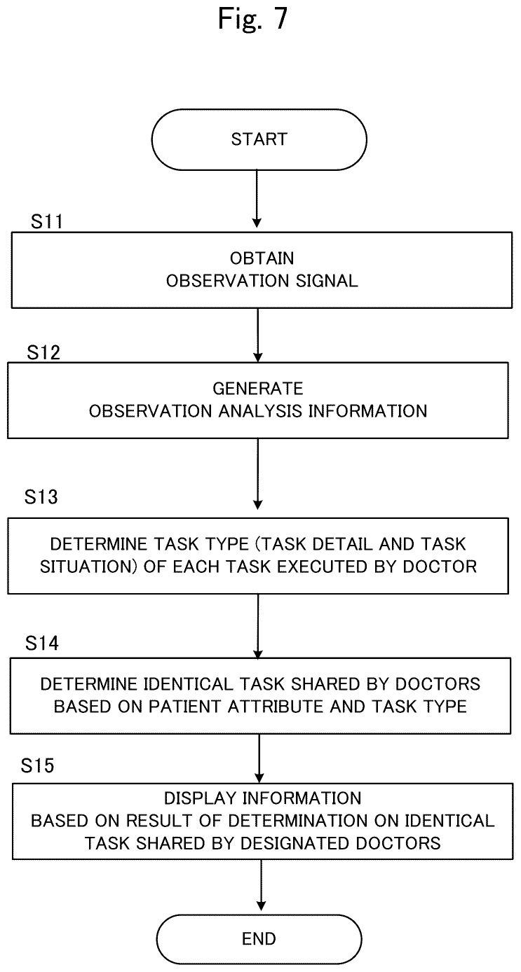

FIG. 7 is an exemplary flowchart to be executed by the task management device 1 according to the first example embodiment. The task management device 1 repeatedly executes the process of the flowchart.

First, the task management device 1 obtains the time-series observation signal S3 supplied from the observation device 5 (step S11). The task management device 1 may store the obtained observation signal S3 in the storage device 4. Then, the task management device 1 analyzes the obtained observation signal S3, and generates the observation analysis information D1 indicating a result of the analysis of the observation signal S3 in time series (step S12). Then, the task management device 1 stores the generated observation analysis information D1 in the storage device 4.

Then, the task management device 1 determines a task type (specifically, type of task details and task situation) of each task executed by the doctor based on the observation analysis information D1 (step S13). The task management device 1 recognizes the patient as a subject of each task executed by the doctor based on the patient charge information in the patient information D4, and stores, in the storage device 4, the task type information D3 in which the doctor as the task entity, the patient, the task type, and the time stamp are associated with each other for each task executed by the doctor.

Next, the task management device 1 determines the identical task shared by the doctors based on the patient attribute and the task type (step S14). In this case, the task management device 1 recognizes the patient attribute and the task type for each task based on the task type information D3 and the patient attribute information in the patient information D4, and determines the identical task shared by the doctors to be compared based on the patient attribute and the task type. In this case, the task management device 1 may determine the identical task shared by the doctors designated by the user input using the input device 2, or may determine the identical task for combinations of all the doctors whose task types are registered in the task type information D3. Then, the task management device 1 stores the identical task determination information D5 indicating a result of the determination regarding the identical task in the storage device 4.

Then, the task management device 1 displays information based on the result of the determination regarding the identical task shared by the doctors designated by the user input (step S15). In this case, the task management device 1 generates the display control signal S2 with reference to the identical task determination information D5 and the task type information D3 relevant to the pair of doctors designated by the user input, for example, and supplies the generated display control signal S2 to the display device 3, thereby causing the display device 3 to display the operating efficiency comparison screen image illustrated in FIG. 6. As a result, the task management device 1 may accurately present the tasks that may be effectively compared in the review of the operating efficiency and the like.

### (4) Modified Examples

Next, modified examples of the example embodiment described above will be described. The following modified examples may be applied to the example embodiment described above in any combination.

### First Modified Example

The task management device 1 may determine the identical task shared by equal to or more than three doctors designated by the user input.

In that case, the task management device 1 specifies the task shared by all the three or more doctors designated based on the input signal S1 output from the input device 2 operated by the user. In that case, the task management device 1 determines, as the identical task, the task having commonality in the task type and in the patient attribute for the three or more doctors. Then, for each of the three or more doctors, the task management device 1 displays a bar graph indicating a required time per task executed by each doctor on the operating efficiency comparison screen image, and also displays the required times for the identical tasks in association with each other by color-coding display, arrow display, or the like.

In this manner, according to the present modified example, the task management device 1 may determine the identical task shared by doctors even when equal to or more than three doctors to be compared are designated by a user input, and may suitably present a result of the determination to the user.

### Second Modified Example

The task entity is not limited to a doctor, and may be any task entity whose target of a task is a person.

Examples of the task entity other than the doctor include a care worker who deals with a person requiring nursing care, a beauty specialist and other service providers who deal with clients, and an educator such as a teacher who deals with students. Even in that case, the task management device 1 may determine the identical task shared by the task entities based on the task type and the task subject attribute, and may present a result of the determination.

### Third Modified Example

The task entity to be compared is not necessarily a single person, and may be an organization to which one or a plurality of persons belong. For example, when an organization to which doctors belong is to be compared, the organization may be in units of clinical departments, in units of teams, or in units of hospitals.

In that case, for example, the task management device 1 determines an attribute of a subject (e.g., patient) of a task and a task type of each task executed by a person (e.g., doctor) belonging to the organization for each organization to be compared. Then, the task management device 1 determines the identical task shared by the organizations. The task management device 1 calculates an average required time for each task when there are tasks having commonality in the task type and in the attribute of the subject of the task in the organization. Then, for each organization to be compared, the task management device 1 displays a bar graph indicating a required time for each task on the operating efficiency comparison screen image, and also displays the required times for the identical tasks determined between the organizations to be compared in association with each other.

In this manner, according to the present modified example, the task management device 1 may determine the identical task shared by organizations designated by the user even when the organizations are to be compared, and may suitably present a result of the determination to the user.

Here, the present modified example will be described in a supplemental manner. In the present modified example, the user may set an issue to be addressed by an organization in consideration of a management index (such as index representing income and expenditure) of the organization to be compared. For example, in a first organization and a second organization to be compared, even when a time required for a first task, which is the identical task, is longer in the second organization than in the first organization, the difference in the time required for the first task may be considered not to be a disadvantage of each organization if the management indexes of the first organization and the second organization are the same. Thus, in that case, the user does not consider the first task as an issue to be addressed by the second organization. On the other hand, when the time required for the first task, which is the identical task, is longer in the second organization than in the first organization and the management index of the first organization is better than that of the second organization, the prolongation of the time required for the first task in the second organization may be considered to be a cause of deterioration in the management index. Thus, in that case, the user sets the required time of the first organization regarding the first task as a target required time of the second organization regarding the first task.

### Second Example Embodiment

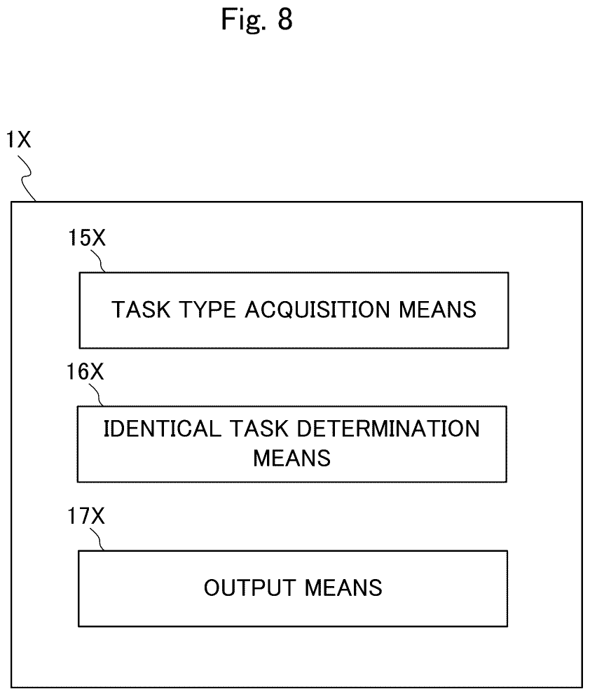

FIG. 8 is a block diagram of the task management device 1X according to the second example embodiment. Examples of the task management device 1X include the task management device 1 according to the first example embodiment. The task management device 1X mainly includes a task type acquisition means 15X, an identical task determination means 16X, and an output means 17X. The task management device 1X may be configured by a plurality of devices.

The task type acquisition means is configured to obtain task type information, which is information indicating a task type related to each task executed in time series by a task entity that deals with a subject as a target of the task. The task type acquisition means 15X may generate the task type information based on signals output by sensors or may receive (including read out from a storage device) the preliminarily-generated task type information. Examples of the task type acquisition means 15X include the task type determination means 15 according to the first example embodiment.

The identical task determination means 16X is configured to determine an identical task shared by a first task entity and a second task entity based on the task type information and attribute information of the subject. Examples of the identical task determination means 16X include the identical task determination unit 16 according to the first example embodiment.

The output means 17X is configured to output information related to the identical task. Examples of “output information” herein include not only “display or audio-output information” and “cause another device to display or audio-output information” but also “transmit information to another device” and “store information”. Examples of the output means 17X include the output unit 17 according to the first example embodiment.

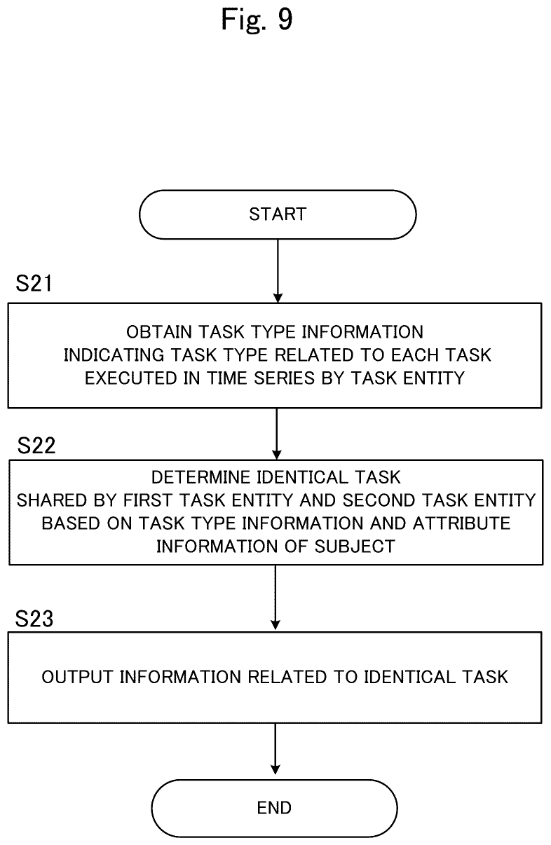

FIG. 9 illustrates an example of the flowchart executed by the task management device 1X according to the second example embodiment. The task type acquisition means obtains task type information, which is information indicating a task type related to each task executed in time series by a task entity that deals with a subject as a target of the task (step S21). The identical task determination means 16X determines an identical task shared by a first task entity and a second task entity based on the task type information and attribute information of the subject (step S22). The output means 17X outputs information related to the identical task (step S23).

According to the second example embodiment, the task management device 1X determines a comparable identical task among task entities to suitably output the determination result.

In the example embodiments described above, the program is stored by any type of a non-transitory computer-readable medium (non-transitory computer readable medium) and can be supplied to a control unit or the like that is a computer. The non-transitory computer-readable medium include any type of a tangible storage medium. Examples of the non-transitory computer readable medium include a magnetic storage medium (e.g., a flexible disk, a magnetic tape, a hard disk drive), a magnetic-optical storage medium (e.g., a magnetic optical disk), CD-ROM (Read Only Memory), CD-R, CD-R/W, a solid-state memory (e.g., a mask ROM, a PROM (Programmable ROM), an EPROM (Erasable PROM), a flash ROM, a RAM (Random Access Memory)). The program may also be provided to the computer by any type of a transitory computer readable medium. Examples of the transitory computer readable medium include an electrical signal, an optical signal, and an electromagnetic wave. The transitory computer readable medium can provide the program to the computer through a wired channel such as wires and optical fibers or a wireless channel.

The whole or a part of the example embodiments (including modifications, the same shall apply hereinafter) described above can be described as, but not limited to, the following Supplementary Notes.

### Supplementary Note 1

A task management device comprising:
a task type acquisition means for obtaining task type information, which is information indicating a task type related to each task executed in time series by a task entity that deals with a subject as a target of the task;
an identical task determination means for determining an identical task shared by a first task entity and a second task entity, based on the task type information and attribute information of the subject; and
an output means for outputting information related to the identical task.

### Supplementary Note 2

The task management device according to Supplementary Note 1, wherein the output means outputs information related to a required time required for the identical task by each of the first task entity and the second task entity.

### Supplementary Note 3

The task management device according to Supplementary Note 2, wherein the output means displays a required time of the task executed by each of the first task entity and the second task entity, and displays the required time of the task determined to be the identical task in association with each other.

### Supplementary Note 4

The task management device according to Supplementary Note 1, wherein the task type includes a type related to a task situation, which is a scene in which the task is performed, and to a task detail, which is a detail of the task.

### Supplementary Note 5

The task management device according to Supplementary Note 1, wherein the identical task determination means determines, as the identical task, the task having commonality in the task type and in an attribute of the subject indicated by the attribute information among a plurality of the tasks executed by the first task entity and the second task entity.

### Supplementary Note 6

The task management device according to Supplementary Note 5, wherein the identical task determination means determines whether the attribute of the subject is common for a type of the attribute determined based on the task type.

### Supplementary Note 7

The task management device according to Supplementary Note 1, wherein
the task type is represented by a plurality of levels of granularity, and
the identical task determination means determines the identical task based on the granularity selected from the plurality of levels of granularity.

### Supplementary Note 8

The task management device according to Supplementary Note 1, wherein
the task type is represented by a plurality of levels of granularity, and
the identical task determination means excludes, from a candidate for the identical task, the task different in the task type at a coarsest granularity among a plurality of the tasks executed by the first task entity and the second task entity.

### Supplementary Note 9

The task management device according to Supplementary Note 1, wherein the identical task determination means determines the identical task among the first task entity, the second task entity, and one or a plurality of the task entities other than the first task entity and the second task entity.

### Supplementary Note 10

The task management device according to Supplementary Note 1, wherein the task entity includes an organization that performs a task of dealing with the subject.

### Supplementary Note 11

The task management device according to Supplementary Note 1, wherein the subject includes a patient, and the task entity includes a doctor or an organization to which the doctor belongs.

### Supplementary Note 12

The task management device according to Supplementary Note 1, wherein the task type acquisition means generates the task type information based on an observation signal output from an observation device that observes the task by the task entity.

### Supplementary Note 13

A task management method for causing a computer to perform:
obtaining task type information, which is information indicating a task type related to each task executed in time series by a task entity that deals with a subject as a target of the task;
determining an identical task shared by a first task entity and a second task entity based on the task type information and attribute information of the subject; and
outputting information related to the identical task.

### Supplementary Note 14

A storage medium storing a program for causing a computer to perform a process comprising:
obtaining task type information, which is information indicating a task type related to each task executed in time series by a task entity that deals with a subject as a target of the task;
determining an identical task shared by a first task entity and a second task entity based on the task type information and attribute information of the subject; and
outputting information related to the identical task.

While the invention has been particularly shown and described with reference to example embodiments thereof, the invention is not limited to these example embodiments. It will be understood by those of ordinary skilled in the art that various changes in form and details may be made therein without departing from the spirit and scope of the present invention as defined by the claims. In other words, it is needless to say that the present invention includes various modifications that could be made by a person skilled in the art according to the entire disclosure including the scope of the claims, and the technical philosophy. All Patent and Non-Patent Literatures mentioned in this specification are incorporated by reference in its entirety.

### DESCRIPTION OF REFERENCE NUMERALS

1, 1A, 1X Task management device
2 Input device
3 Display device
4 Storage device
5 Observation device
100, 100A Task management system

What is claimed is:

1. A task management device comprising:
at least one memory configured to store instructions; and
at least one processor configured to execute the instructions to:
obtain information on a neural network model trained to determine a task type, wherein the information includes parameters regarding a layer structure, a neuron structure of each layer, a number of filters and a filter size in each layer, a weight of each element of each filter;
determine the task type related to each task executed in time series by a task entity that deals with a subject as a target of the task, based on the neural network model;
obtain task type information, which is information indicating the task type;
determine an identical task shared by a first task entity and a second task entity, based on the task type information and attribute information of the subject; and
output information related to the identical task including information related to a required time required for the identical task by each of the first task entity and the second task entity,
wherein the at least one processor is configured to execute the instructions to display the required times of the tasks by the first task entity and the required times of the tasks by the second task entity, while displaying the required time of the identical task by the first task entity in association with the required time of the identical task by the second task entity.

2. (canceled)

3. (canceled)

4. The task management device according to claim 1, wherein the task type includes a type related to a task situation, which is a scene in which the task is performed, and to a task detail, which is a detail of the task.

5. The task management device according to claim 1, wherein the at least one processor is configured to execute the instructions to determine, as the identical task, the task having commonality in the task type and in an attribute of the subject indicated by the attribute information among a plurality of the tasks executed by the first task entity and the second task entity.

6. The task management device according to claim 5, wherein the at least one processor is configured to execute the instructions to determine whether the attribute of the subject is common for a type of the attribute determined based on the task type.

7. The task management device according to claim 1, wherein
the task type is represented by a plurality of levels of granularity, and
the at least one processor is configured to execute the instructions to determine the identical task based on the granularity selected from the plurality of levels of granularity.

8. The task management device according to claim 1, wherein
the task type is represented by a plurality of levels of granularity, and
the at least one processor is configured to execute the instructions to exclude, from a candidate for the identical task, the task different in the task type at a coarsest granularity among a plurality of the tasks executed by the first task entity and the second task entity.

9. The task management device according to claim 1, wherein the at least one processor is configured to execute the instructions to determine the identical task among the first task entity, the second task entity, and one or a plurality of the task entities other than the first task entity and the second task entity.

10. The task management device according to claim 1, wherein the task entity includes an organization that performs a task of dealing with the subject.

11. The task management device according to claim 1, wherein the subject includes a patient, and the task entity includes a doctor or an organization to which the doctor belongs.

12. The task management device according to claim 1, wherein the at least one processor is configured to execute the instructions to generate the task type information based on an observation signal output from an observation device that observes the task by the task entity.

13. A task management method for causing a computer to perform:
obtain information on a neural network model trained to determine a task type, wherein the information includes parameters regarding a layer structure, a neuron structure of each layer, a number of filters and a filter size in each layer, a weight of each element of each filter;
determine the task type related to each task executed in time series by a task entity that deals with a subject as a target of the task, based on the neural network model;
obtaining task type information, which is information indicating a task type related to each task executed in time series by a task entity that deals with a subject as a target of the task;
determining an identical task shared by a first task entity and a second task entity based on the task type information and attribute information of the subject;
outputting information related to the identical task including information related to a required time required for the identical task by each of the first task entity and the second task entity; and
displaying the required times of the tasks by the first task entity and the required times of the tasks by the second task entity, while displaying the required time of the identical task by the first task entity in association with the required time of the identical task by the second task entity.

14. A non-transitory computer readable storage medium storing a program for causing a computer to perform a process comprising:
obtain information on a neural network model trained to determine a task type, wherein the information includes parameters regarding a layer structure, a neuron structure of each layer, a number of filters and a filter size in each layer, a weight of each element of each filter;
determine the task type related to each task executed in time series by a task entity that deals with a subject as a target of the task, based on the neural network model;
obtaining task type information, which is information indicating the task type;
determining an identical task shared by a first task entity and a second task entity based on the task type information and attribute information of the subject;
outputting information related to the identical task including information related to a required time required for the identical task by each of the first task entity and the second task entity; and
displaying the required times of the tasks by the first task entity and the required times of the tasks by the second task entity, while displaying the required time of the identical task by the first task entity in association with the required time of the identical task by the second task entity.
